(19) United States
(12) Patent Application Publication
(10) Pub. No.: US 2019/0131018 A1
(43) Pub. Date: May 2, 2019

(54) SYSTEM AND METHOD FOR ANALYZING DISEASES

(71) Applicant: R Zero Tracing, Inc., Ridgeland, MS (US)

(72) Inventor: KEN SONES, Ridgeland, MS (US)

(73) Assignee: R Zero Tracing, Inc., Ridgeland, MS (US)

(21) Appl. No.: 16/172,351

(22) Filed: Oct. 26, 2018

Related U.S. Application Data
(60) Provisional application No. 62/577,541, filed on Oct. 26, 2017.

(51) Int. Cl. G16H 50/80; G06F 15/18; G16H 10/20

(52) U.S. Cl. CPC G16H 50/80; G16H 50/20; G16H 10/20; G06F 15/18

(57) ABSTRACT

We disclose herein a system and method to help health scientists identify how diseases develop. This software toolkit contains tools for analyzing spatiotemporal factors pertaining to health in real-time and retrospect for individuals and populations. With the individualized location tracking and hyperlocal environmental monitoring, the system could help prevent diseases and find causes of diseases of unknown origin

### CROSS-REFERENCE TO RELATED APPLICATION

This application claims the benefit of U.S. Provisional Application No. 62/577,541 filed Oct. 26, 2017, which is incorporated herein by reference in its entirety.

### FIELD OF THE INVENTION

The present invention is generally directed toward a system and method for analyzing disease transmission patterns.

### BACKGROUND OF THE INVENTION

It is widely known that many health issues can be attributed to environmental factors. Asthma, for example, is triggered by, among other environmental factors, high amounts of pollen or other fine particulate matter. In another study, Geoffrey Martin of the University of Cincinnati published an article in 2013 suggesting lightning strikes could trigger migraines. Environmental exposure to blue-green algae has even been attributed as a cause of ALS.

It would be beneficial to have a tool that could help track disease development and symptoms in relationship to environmental factors and alert affected users. For example, alerting asthma patients that there are high levels of pollen could help them avoid extended time outdoors, which may reduce their asthma attacks. Some tools like this exist already.

Other diseases are caused by highly contagious pathogens. For example, smallpox is incredibly infectious and a huge percentage of Americans are susceptible. By identifying and quarantining people exposed to infectious smallpox particles before they become infectious, officials could prevent an epidemic spread. However, the standard epidemiological method for studying the disease progression through a society is to interview the infected people. This method relies on trying to recreate a timeline from memory, which may be unreliable.

It would be beneficial to have a tool that could recreate an individual's exact location history to provide accurate information to doctors and public health officials. For example, Tuberculosis can be latent in a person for months or years. If a doctor could look at a patient's location history and determine where the infection began, he could prevent future cases from developing.

### SUMMARY OF THE INVENTION

We disclose herein a system and method to help scientists identify how diseases develop and environmental factors that may result in symptoms. This software toolkit contains tools for analyzing spatiotemporal factors pertaining to health in real-time and retrospect for individuals and populations.

### BRIEF DESCRIPTION OF THE DRAWINGS

Further advantages of the invention will become apparent by reference to the detailed description of preferred embodiments when considered in conjunction with the drawings:

FIG. 1 depicts a layout of the system architecture.

FIG. 2 depicts a graphical health timeline.

FIG. 3 depicts a schematic of Timeline Alignment.

FIG. 4 depicts a representation of the modules in the system.

FIG. 5 depicts a representation of the pathogen exposure.

FIG. 6 depicts an exemplary screenshot of the toolkit in use

FIG. 7 depicts another exemplary screenshot of the toolkit in use.

FIG. 8 depicts an exemplary quantitative health questionnaire utilized in the system.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

We disclose herein tools to study how space and time affect disease development and symptoms and methods for alerting affected people. This could be used in a variety of ways, such as: to manage diseases triggered by spatiotemporal factors; to prevent the spread of highly contagious pathogens; and to search for the root cause of diseases of unknown origin. Any of these use cases would yield immense societal value.

Our platform combines geospatial data, spatiotemporal data, and health data to identify the potential source of health conditions. The platform combines data through software that analyzes the datasets and then uses statistical analyses to identify significant variables. To get accurate analyses, our system uses individuals' or a population's health records, individuals' location histories, remotely sensed environmental data, hyperlocal environmental data from the disclosed sensors, and other geospatial features.

In one embodiment, the disclosed platform is a web application that allows for different types of statistical analyses in a modular manner. The basic component of this application is a web interface that loads and visualizes a layer of location histories (including from mobile device location history) and different layers of accessory spatial data (keypoints, temperature rasters, vectors, etc.)

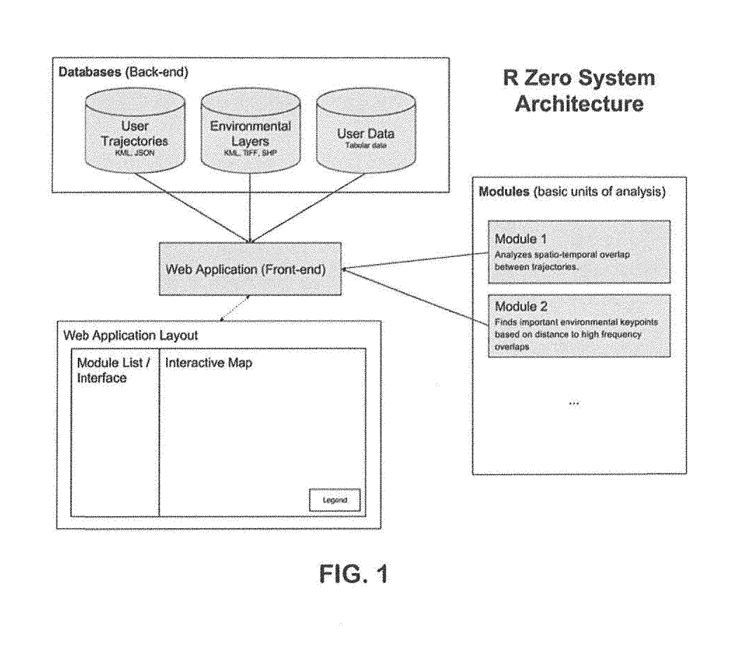

This data is operated on by modules that each performs a specific type of analysis. Each module developed for the application allows for a different type of analysis. In the current embodiment, each module exists as one or two pieces—a JavaScript library and a back-end API (if necessary). The separation of module logic from visualization component is preferred, as different types of analyses require vastly different computation resources. For example, a module that trains deep recurrent neural networks to identify periodic behavior in groups of user trajectories will require dedicated computing resources, and should not run on the same system that serves the web interface. Additionally, not all customers will need access to the same set(s) of modules. See FIG. 1 for a general visualization of the system architecture.

Additionally, we disclose products that complement the web application. These include environmental sensors to collect hyperlocal environmental data that can be deployed in a variety of ways, including as weather stations or mobile transmitters; data scrapers to mine information from public sources, such as NASA's satellite portal; and native mobile applications that can be used to collect location history data.

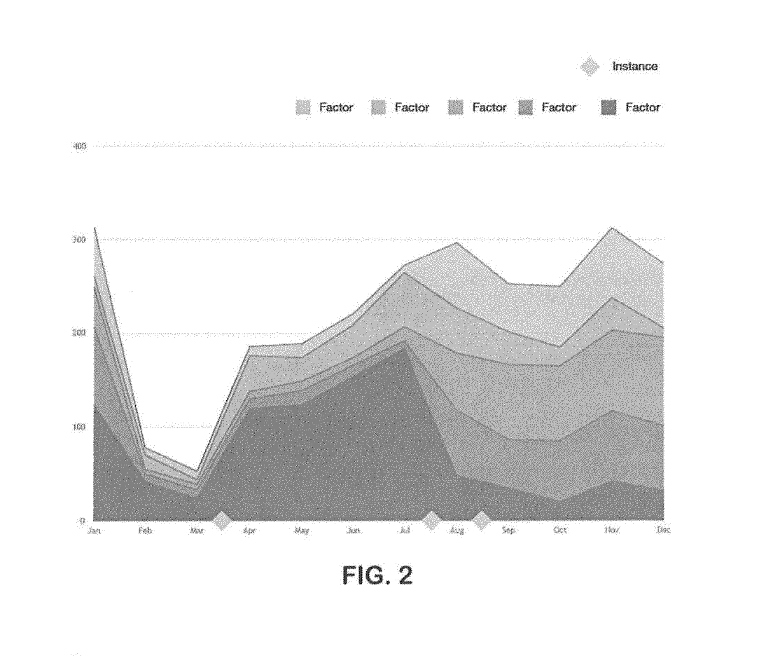

We further disclose a customizable health timeline. As shown in FIG. 2, the health timeline is a graphical way to view a patient's health history in conjunction with environmental factors. In the graph, the diamond shaped marks along the x axis represent the patient's health events and the y axis represents environmental variables. Among other options, the user may change the values and axes of the graph.

It should be appreciated that the system and software toolkit can be developed using any software language, social API, or programmable architecture such as HTML/CSS, PHP, MySQL, Twitter Bootstrap, JS, Python, C/C++, Arduino, Raspberry Pi, Keras, Tensorflow, Scikit-Learn, MongoDB, Docker and Swift.

The Crossings Engine

The Crossings Engine, our name for the web application and technology surrounding it, is the core of the disclosed software platform. The disclosed system combines individual location histories and environmental data from satellites and sensors using this novel Crossings Engine. The Crossings Engine is trained on a library of disease modules to identify potential sources of conditions based on location and environmental data. This allows public health officials to identify sources of outbreaks in near real time.

Web Application Architecture

The web application consists of two parts: a back-end Python server that hosts the static web content and responds to API calls from the web application and the front-end web application that allows a user to interact with data to use modules to analyze it. This design is based around the modular concept described previously, where a single module contains the functionality for performing a basic unit of analysis on data.

In the absence of any modules, the web application provides:

Loading and unloading a list of trajectory files as a single layer

Loading and unloading a list of keypoint files as a single layer

Displaying the keypoints and trajectories graphically, in a GUI similar to other modern mapping software

Highlighting a single trajectory when hovered over with the mouse

In a potential embodiment of the architecture of the application, the file run_keras_server.py contains the majority of the functionality of the application. The load_model( ) function loads in the saved model that was trained on the initial dataset, compiles it, loads in the weights, and loads in the tokenizer. The prepare_data(data) function receives data, uses the tokenizer to convert the texts in the data to sequences, pads the sequences, and returns the padded sequences.

There are three routes set up after the main methods: The first route is for the home page that gives the user an option to navigate to the loadFile page. The second route is the loadFile page. There, the user can upload a j son file containing the patient notes to receive the trajectory prediction. The third route is the prediction route, which receives a flask post request and will return the trajectory predictions. This route may be contacted via API or through our site's User Interface.

Trajectory Slicing and Dicing

A key challenge in analyzing spatiotemporal factors that contribute to health is analyzing where people go in a relevant way. To do this, the disclosed platform inputs user trajectories, reduces them to relevant scopes, and further splits them into useful periods. The trajectories come from a variety of places, such as our app and Google Timeline (https://www.google.com/maps/timeline). These trajectories are typically GeoJSON or KML files, but are sometimes in other formats.

The Crossings Engine considers a few parameters for reduction; based on the disease under examination and date of diagnosis, trajectories typically can be reduced to a matter of weeks or days (rather than years). This is typical, but not guaranteed. For example, consider Legionnaire's Disease and Tuberculosis. Legionnaire's Disease is almost always diagnosed within 20 days of exposure to legionella. Unlike Legionnaire's Disease, Tuberculosis can take years to diagnose, this means the Crossings Engine might need to take in years of location history data to provide valuable output concerning tuberculosis.

The Crossings Engine can then process data into trajectory units in two ways: by day, where the first location-time point of a given day is the beginning point for the trajectory and the last location-time point of the day ends the trajectory, or by “idle threshold” time, where trajectories are created based on contiguous blocks of movement.

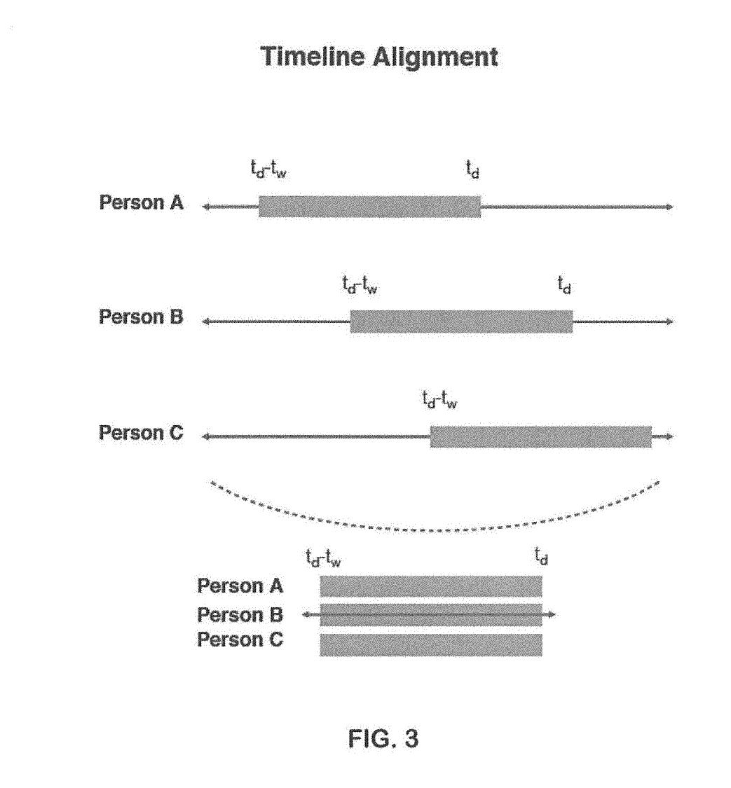

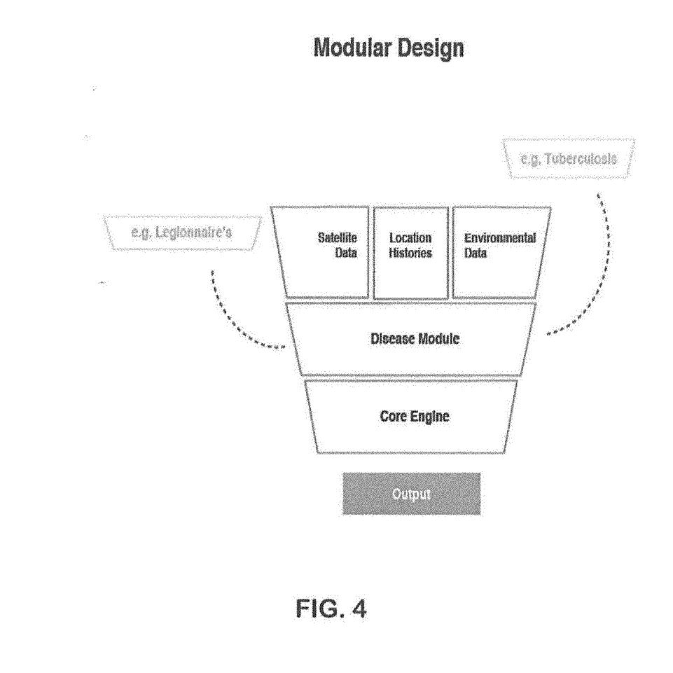

As will be appreciated from FIG. 3, the backend slices and arranges timelines based on the time of diagnosis t_{d} and the disease time window t_{w}. FIG. 4 shows that the Crossings Engine has disease modules that calibrate key variables, such as the time window to examine or the pathogen lifespan. Because of the Crossings Engine's modular design, these parameters can be changed easily and tweaked endlessly.

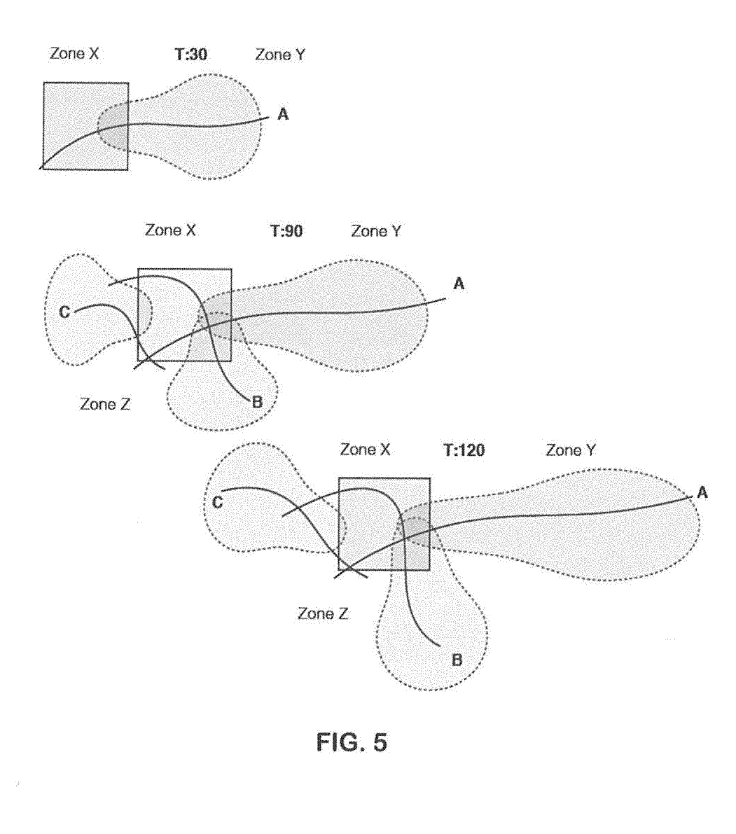

As will be appreciated from FIG. 5, our Crossings Engine Disease Modules allow researchers to investigate specific outbreaks or diseases with specific parameters. For example, the mysterious Pathogen X has the following disease module parameters: Continuous Source Outbreak, Person-to-Person Infectious, Disintegrating Bounding Box. It can then be calculated that:

From t=0 to tₑₓₚₒₛᵤᵣₑ<60, the chance of infection if exposed to the pathogen is 100%.

From t=60 to tₑₓₚₒₛᵤᵣₑ<120, the chance of infection if exposed to the pathogen is 50%.

From tₑₓₚₒₛᵤᵣₑ=120 onward, the chance of infection if exposed to the pathogen is 0%, and

Exposed subjects are immediately infected and infectious.

Once a module and data set are loaded into the Crossing Engine, a machine-learning algorithm identifies the spatiotemporal factors that may be contributing to the development of conditions or diseases.

Keypoint Data

Geographic keypoints are a second important data set for analyzing how spatiotemporal factors contribute to health outcomes. There are many different types of geographic keypoints and many data sources that provide them. For example, restaurants, supermarkets, parks, and highway exits are all common geographic keypoints in geospatial information system analysis. OpenStreetMap is an open-source effort that provides these and many other key points around the world for free. The Crossings Engine allows users to input huge sets of keypoints and use them for analysis, including custom data and data from public sources, such as OpenStreetMap.

Environmental Sensing

Environmental variables provide an important and challenging dimension to spatiotemporal health analysis. Environmental data is collected globally from satellite-borne sensors and locally from weather stations. Much of this data is publicly accessible; NASA and other government entities publish data they collect on web portals regularly. Some other websites, such as Accuweather, allow users to upload data from private weather stations to create large datasets. There are some limits to the effectiveness of this environmental data; for some measurements, rural areas lack local data and are typically represented by projections from the nearest urban center. In addition to collecting data from publicly accessible sources, the disclosed platform provides a way to collect hyperlocal environmental data via environmental transmitters.

Environmental Transmitters

To collect environmental data from rural Mississippi locations, our team created two sets of transmitters. One set of transmitters uses a WiFi connection to send sensor records to a web database through an API. The other set of transmitters uses a GSM (cell phone) data connection to send sensor records to a web database through an API. The data connection is the primary difference between the two embodiments.

The WiFi-based sensor consists of an Arduino MEGA, a Raspberry Pi, an optional GPS unit, and one or more environmental sensors. This transmitter is designed either to be stationary or mounted on a vehicle (in which case a GPS unit would be required). The transmitter uses the Arduino microcontroller to read the GPS and any environmental sensors. The Arduino then sends this data to the Raspberry Pi, which is listening to the Arduino via a USB port and a Python script. The Python Script then processes the data, storing it locally. Another Python script checks for a WiFi connection regularly and, when connected to the internet via WiFi, retrieves all locally stored data, sends it to a web database through a series of API calls, then dumps the local copies of the readings. In theory, this transmitter could store many gigabytes of data. The Raspberry Pi's operating system boots from a microSD card, which also keeps the locally stored data. In the current version of the transmitter, this is a 32 GB microSD card, which would allow for many days of regular environmental readings before encountering memory restrictions.

The GSM-based sensor consists of an Arduino Uno, and Adafruit FONA (GPS and GSM Modules), a GSM SIM card, a GPS Antenna, a GSM antenna, a battery, and one or more environmental sensors. This transmitter is designed to be mounted on a vehicle and powered by a fuse tap connected to the vehicle's fuse panel. As a proof-of-concept, we mounted transmitters on a fleet of buses in two counties in northern Mississippi. The transmitter uses the Arduino microcontroller to read the GPS and the environmental sensor(s), prepare an API-compatible URL, and interact with the FONA module. The FONA processes the URL and reads the data from the API (which typically responds with “OK”—meaning the data was successfully processed by the API). After attempting to send the collected data, the Arduino waits for a programmable number of seconds and then repeats the operation. In the same loop, the Arduino verifies that it has a GPS location lock, a cellular network connection, and a GSM data connection. If any of these fail, the Arduino prioritizes reconnecting before reading the sensor again. This transmitter is mounted inside a 3″ by 5″ case, with the environmental sensors mounted to the outside of the case to allow for sufficient exposure to the environment.

Modules

As described previously, the Crossings Engine has a modular design, where different computational modules add functionality to the basic web application. In this section, the first three modules are described in detail. In general, a module consists of a back-end and a front-end component. The backend code is responsible for registering API endpoints to interact with the front-end code and doing the bulk of the computational analysis. The front-end part of a module is responsible for initializing the computation (via an init( ) method), drawing GUI elements, and handling clicks on the module to execute functionality. The purpose of this separation is to decouple module code from the rest of the functionality of the web application as much as possible. This currently is a key architectural focus of the platform.

Trajectory Similarity Module

The trajectory similarity module compares all of the trajectories loaded into the web application and groups them into clusters of “similar behavior.” Specifically, the module uses the discrete Frechet distance to calculate all trajectory pairs' “similarity.” The discrete Frechet distance between two trajectories is 0 if the trajectories are identical, and grows as the trajectories become more dissimilar. The module uses the computed distances to perform a clustering of the trajectories with agglomerative tree based clustering. This clustering algorithm requires a set parameter of how many clusters to find. This module performs the analysis multiple times for different numbers of clusters in the range [2,10] in order to find the “best” number of clusters. The “goodness” of a particular cluster is evaluated using the silhouette value, which is larger for better clusterings. This module returns the clustering that maximizes the silhouette score.

Trajectory-Keypoint Finder Module

The trajectory-keypoint finder module analyzes which keypoints are “most visited” by the trajectories in the study area. To do this, each keypoint is buffered by some distance then intersected with each trajectory. The number of trajectories that the keypoint intersects with is considered to be the number of times it is “visited” by the trajectory set. The module returns the number of times each keypoint is visited and colors the keypoints in the GUI accordingly.

This module is both useful for some public health epidemiology contexts and an example of how to integrate keypoint and trajectories interworking into the module based analysis framework and could be extended.

Firetower Module

The “Firetower” module analyzes trajectory commonality from the trajectory set. To do this, each trajectory is buffered by some distance to create a polygon and then compared to all other trajectories. The module then creates sets where polygons overlap n number of times. This is a difficult problem because there are a factorial number of intersection operations to perform. Per trajectory, brute force calculation would create an exponentially more difficult (and, more important, computationally expensive) operation. To control for this complication, this module calculates overlaps at (n Choose x) and propagates those. This module returns polygons of overlapping trajectory buffers and then colors them on the GUI based on the number of overlaps.

This module is both useful for some public health epidemiology contexts and an example of how to create trajectory-trajectory comparison within the module based analysis framework.

### Usage Examples

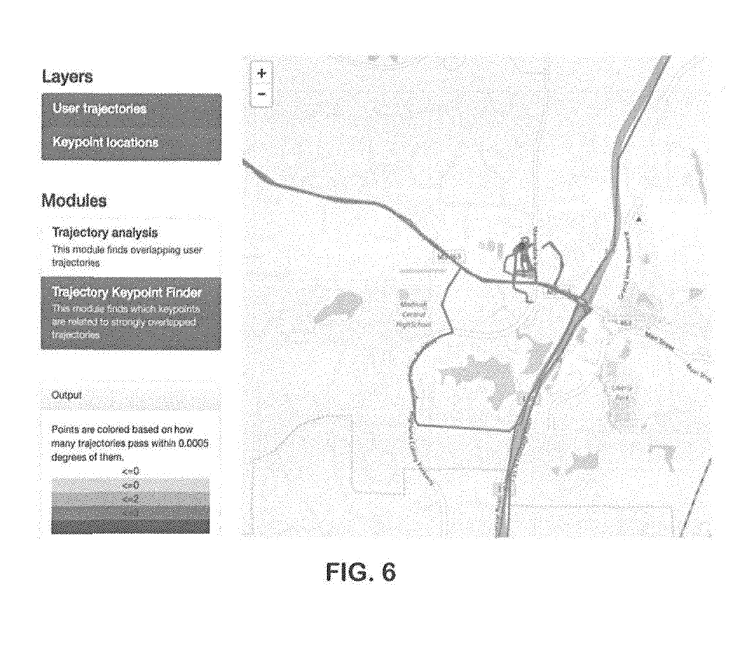

The Crossings Engine takes a set of location histories, environmental data, key points, and a disease module to identify potential sources of infection. FIG. 6 shows an example of the information that can be gleaned from this tool. In this image, the Crossings Engine processed a series of location histories tagged with Legionnaires' Disease and identified key points within Madison, Miss. where the infection could have originated.

We have deployed a suite of environmental sensors across Mississippi. Over the course of this pilot, our sensors have collected over 25,000 environmental readings across more than 200,000 GPS points. The transmitters have been mounted on private vehicles, public school buses, and even drones. After identifying geographic areas with high rates of some condition, these transmitters will allow our team to create highly detailed, hyperlocal pictures of the environment and investigate what environmental factors (if any) are contributing to the condition.

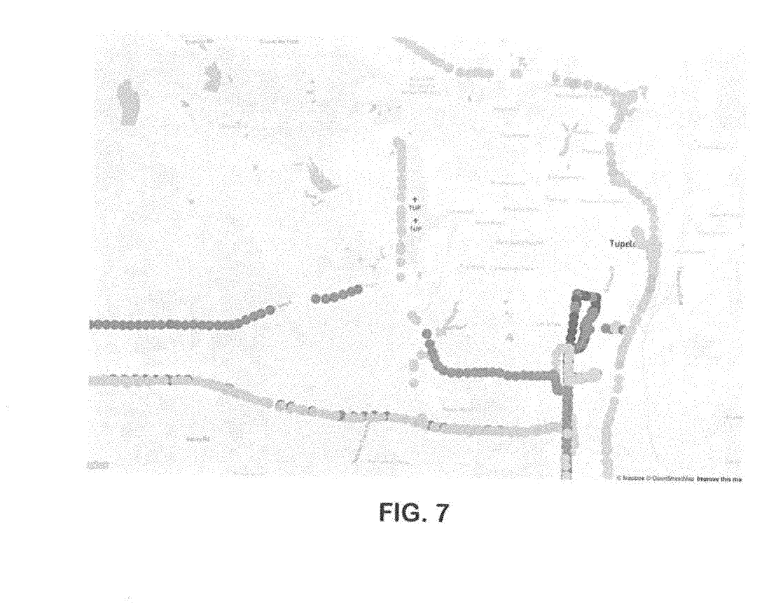

The sensors create hyperlocal maps for many environmental variables. As will be appreciated from FIG. 7, the screenshot shows the VOC levels in Tupelo, Miss. along major roads. The yellow areas are VOC levels that could be dangerous over an extended period of time.

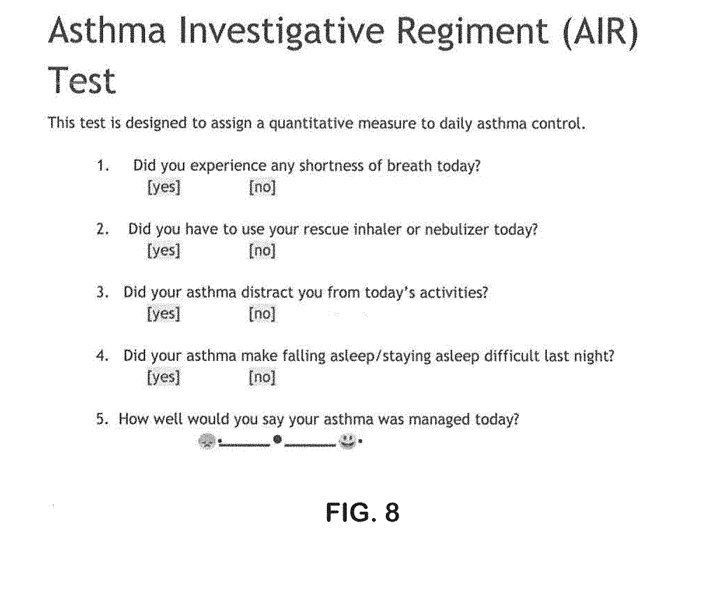

As shown in FIG. 8, data is obtained directly from patients via questionnaires and tracked over time to provide quantitative measures of daily health and specific health-related issues, such as asthma.

We anticipate that this disclosed invention has several potential uses. For example it could be useful to Public Health Organizations as an outbreak investigation and containment tool, to health insurers as an actuarial investigation tool, and/or to financial institutions to gain insights for site selection and financial modeling. Schools and companies could be interested in the tool for tracking and increasing attendance. It can also be used as a diagnostic tool.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

We claim:

1. A software to help health scientists identify how diseases develop comprising tools for analyzing spatiotemporal factors pertaining to health in real-time and retrospect for individuals and populations.

2. The software of claim 1 wherein a machine learning algorithm identifies the spatiotemporal factors that may be contributing to the development of conditions or diseases.
